(19) United States
(12) Patent Application Publication
(10) Pub. No.: US 2025/0032397 A1
(43) Pub. Date: Jan. 30, 2025

(54) METHOD FOR COLORING HUMAN HAIR, COMPRISING THE APPLICATION OF A DYE HAVING AMINOSILICONE AND PIGMENT AND SUBSEQUENT IRRADIATION WITH UV/VIS RADIATION

(71) Applicant: Henkel AG & Co. KGaA, Duesseldorf (DE)

(72) Inventors: Ranju Prasad Mandal, Düsseldorf (DE); Constanze Kruck, Grevenbroich (DE); Gabriele Weser, Essen (DE); Daniela Kessler-Becker, Leverkusen (DE)

(21) Appl. No.: 18/910,515

(22) Filed: Oct. 9, 2024

Related U.S. Application Data
(63) Continuation of application No. PCT/EP2023/050416, filed on Jan. 10, 2023.

(30) Foreign Application Priority Data
Apr. 12, 2022 (DE) 102022203646.8

(51) Int. Cl. A61K 8/898; A61Q 5/10

(52) U.S. Cl. CPC A61K 8/898; A61Q 5/10; A61K 2800/43; A61K 2800/4324; A61K 2800/81

(57) ABSTRACT

A method for coloring human hair is provided. The method includes the steps of applying a coloring agent (F) to the hair and irradiating the treated hair for a period of 1 to 60 minutes with UV/VIS radiation in the wavelength range of 200 to 800 nm. The hair coloring agent includes at least one amino-functionalized silicone polymer, and at least one pigment.

### FIELD OF INVENTION

The present application relates to a method for coloring human hair, said method comprising the application of a coloring agent (F) in step (1) and subsequently the irradiation of the hair colored in step (1) for a period of 1 to 60 minutes with UV/VIS radiation in the wavelength range of 200 to 800 nm. The coloring agent (F) contains at least one amino-functionalized silicone polymer (f1) and at least one pigment (f2).

### BACKGROUND

Changing the shape and color of keratin fibers, and in particular human hair, represents an important field of modern cosmetics. To change the hair color, the skilled artisan is familiar with a variety of coloring systems depending upon the coloring requirements. Oxidation coloring agents are typically used for permanent, intense coloring with good fastness properties and good gray coverage. Such coloring agents contain oxidation dye precursors, what are known as developer components, and coupler components, which together form the actual dyes under the influence of oxidizing agents such as, for example, hydrogen peroxide. Oxidation coloring agents are characterized by very long-lasting color results.

When using direct dyes, dyes which are already formed diffuse out of the coloring agent into the hair fiber. In comparison with oxidative hair coloring, the colors obtained with direct dyes have a lower durability and a more rapid washing out. Colors with direct dyes usually remain on the hair for a period of between 5 and 20 hair washes.

The use of color pigments for brief changes in color on the hair and/or the skin is known. Pigments or color pigments are generally understood to mean insoluble coloring substances. These are present undissolved in the form of small particles in the coloring formulation and are only deposited from the outside onto the hair fibers and/or the skin surface. They can therefore generally be removed again without leaving residue by washing a few times with surfactant-containing cleaning agents. Various products of this type by the name of hair mascara are available on the market.

If the user desires particularly long-lasting coloring, the use of oxidative coloring agents has hitherto been the only option. However, despite multiple optimization attempts, an unpleasant ammonia odor or amine odor cannot be completely avoided in oxidative hair coloring. The hair damage that remains associated with the use of the oxidative coloring agents also has a disadvantageous effect on the hair of the user. The search for alternative, high-performance coloring methods is therefore an ongoing challenge. One possible alternative coloring system, which recently has been moving increasingly into focus, is based upon the use of colored pigments.

Coloring with pigments offers various major advantages. Since the pigments are deposited only from the outside to the keratin fibers, and in particular to the hair fibers, the damage associated with the dyeing process is very particularly low. Furthermore, desired colorings that are no longer desired can be removed quickly and easily without residue and therefore offer the user the possibility of returning directly and without great effort to their original hair color. This coloring process is therefore particularly attractive for consumers who do not regularly want to re-color their hair.

In current work, the problem of the low durability of this coloring system has been addressed. In this context, it was found that the wash-fastness of the coloring results obtained with pigments could be greatly improved by combining the pigments with certain amino-functionalized silicone polymers. In addition, the choice of particularly well-suited pigments and pigment concentrations on dark hair achieved a brighter color result so that lightening was even possible with this coloring system, which was possible until now only with oxidative hair treatment agents (bleaching or decolorizing agents).

In order to produce colorings having as high a color stability as possible, various preparations have already been described for the application of the pigments and aminosilicones. However, the shelf life and the wash-fastness that can be obtained from these coloring systems are still in need of improvement. For this reason, possibilities of further improving colorings obtained from pigments with regard to their wash-fastness or their color retention are still sought.

### SUMMARY OF THE INVENTION

The object of the present invention is that of providing a coloring system which has the possibility of fastness properties comparable to oxidative coloring. In particular, the wash-fastness properties should be outstanding, but the use of the oxidation dye precursors normally used for this purpose should be avoided. A technique has been sought that makes it possible to fix the pigments in an extremely durable manner on the hair. When applying the agents in a coloring method, intense coloring results with good fastness properties, and in particular good wash-fastness, and good color retention should be achieved.

The work leading to this invention has now surprisingly shown that colorings produced by application of aminosilicones and pigments on the hair are particularly resistant and resilient if the hair is subjected to artificial irradiation with electromagnetic radiation in the UV/VIS range after the coloring step.

### DETAILED DESCRIPTION OF THE INVENTION

A first subject matter of the present invention is a method for coloring human hair, comprising
(1) applying a coloring agent (F) to the hair which includes
(f1) at least one amino-functionalized silicone polymer, and
(f2) at least one pigment, and
(2) irradiating the hair treated in step (1) for a period of 1 to 60 minutes with UV/VIS radiation in the wavelength range of 200 to 800 nm.

In the context of this invention, tests were carried out in which hair strands were first dyed with a coloring agent (F) containing at least one amino-functionalized silicone polymer (f1) and at least one pigment (f2). Following this coloring step, the hair was then irradiated with an artificial irradiation source with UV/VIS radiation in the wavelength range of 200 to 800 nm. It is shown here that the hair treated in this way was coated with a film of aminosilicone and pigment, which had a much greater resistance compared to non-irradiated hair strands. This improved resistance of the colored hair manifests itself in particular in improved wash-fastness.

### Human Hair

Human hair is to be understood in particular as the head hair, but also eyebrows and eyelashes. Human hair is very particularly preferably understood to be the head hair.

### Coloring Agent

In the context of this invention, the term, “coloring agent,” is used for a coloring of the keratin material, and in particular hair, brought about by use of pigments. In this coloring process, the pigments are deposited as coloring compounds together with the amino-functionalized silicone polymer(s) in a particularly homogeneous, uniform, and smooth film on the surface of the keratin material.

### Application of the Coloring Agent (F) to Hair

In step (1) of the coloring method according to the invention, a coloring agent (F) is applied to the human hair which contains at least one amino-functionalized silicone polymer (f1) and at least one pigment (f2). The coloring agent (F) is a ready-to-use coloring agent.

### Amino-Functionalized Silicone Polymer (f1) in the Coloring Agent

The coloring agent (F) contains at least one amino-functionalized silicone polymer (f1). The amino-functionalized silicone polymer can alternatively also be referred to as aminosilicone or amodimethicone.

Silicone polymers are generally macromolecules with a molecular weight of at least 500 g/mol, preferably at least 1,000 g/mol, more preferably at least 2,500 g/mol, and more preferably at least 5,000 g/mol which comprise repeating organic units.

The maximum molecular weight of the silicone polymer depends upon the degree of polymerization (number of polymerized monomers) and the batch size, and is also determined by the polymerization method. In the context of the present invention, it is preferable if the maximum molecular weight of the silicone polymers is not more than 107 g/mol, preferably not more than 10⁶ g/mol, and particularly preferably not more than 10, g/mol.

The silicone polymers comprise many Si—O repeat units, the Si atoms being able to bear organic groups such as, for example, alkyl groups or substituted alkyl groups. Alternatively, a silicone polymer is therefore also referred to as polydimethylsiloxane.

Corresponding to the high molecular weight of the silicone polymers, these are based upon more than 10 Si—O repeat units, preferably more than 50 Si—O repeat units and particularly preferably more than 100 Si—O repeat units, and very particularly preferably more than 500 Si—O units.

An amino-functionalized silicone polymer is understood to mean a functionalized silicone which bears at least one structural unit with an amino group. The amino-functionalized silicone polymer preferably bears several structural units with at least one amino group in each instance. An amino group is understood to mean a primary amino group, a secondary amino group, and a tertiary amino group. All these amino groups can be protonated in an acidic environment and are then present in their cationic form.

In principle, a good coloring performance with amino-functionalized silicone polymers were achieved if these bear at least one primary, at least one secondary, and/or at least one tertiary amino group. However, intense colorings with the best colorfastness were obtained when an amino-functionalized silicone polymer containing at least one secondary amino group was used in the agent.

In a very particularly preferred embodiment, a method according to the invention is characterized in that a coloring agent (F) is applied to the hair which comprises at least one amino-functionalized silicone polymer (f1) having at least one secondary amino group.

The secondary amino groups(s) may be at different positions of the amino-functionalized silicone polymer. Very particularly good color results were obtained when an amino-functionalized silicone polymer was used that had at least one, and preferably several structural units of the formula (Si-amino).

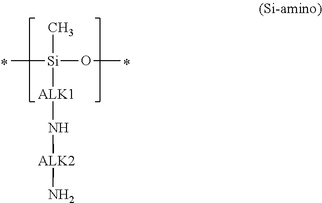

In the structural units of the formula (Si-amino), the abbreviations ALK1 and ALK2 are each independently a linear or branched divalent C₁-C₂₀ alkylene group.

In a further very particularly preferred embodiment, a method according to the invention is characterized in that the coloring agent (F) contains at least one amino-functionalized silicone polymer (f1) which comprises at least one structural unit of formula (Si-amino),

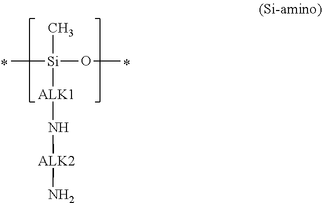

where
ALK1 and ALK2 represent, independently of one another, a linear or branched, divalent C₁-C₂₀ alkylene group.

The positions marked with an asterisk (*) in each case indicate the bond to further structural units of the silicone polymer. For example, the silicon atom adjacent to the asterisk can be bonded to an additional oxygen atom, and the oxygen atom adjacent to the asterisk can be bonded to an additional silicon atom or else to a C₁-C₆ alkyl group.

A divalent C₁-C₂₀ alkylene group can alternatively also be termed a double-bond C₁-C₂₀ alkylene group, which means that each moiety ALK1 or ALK2 can have two bonds.

In the case of ALK1, the silicon atom is bonded to the moiety ALK1, and the second bond is between ALK1 and the secondary amino group.

In the case of ALK2, the secondary amino group bonds with the moiety ALK2, and the second bond is formed between ALK2 and the primary amino group.

Examples of a linear divalent C₁-C₂₀ alkylene group are, for example, the methylene group (—CH₂—), the ethylene group (—CH₂—CH₂—), the propylene group (—CH₂—CH₂—CH₂—), and the butylene group (CH₂—CH₂—CH₂—CH₂—). The propylene group (—CH₂—CH₂—CH₂—) is particularly preferred. Starting at a chain length of 3 C atoms, divalent alkylene groups may also be branched. Examples of branched, divalent C₃-C₂₀ alkylene groups are (—CH₂—CH(CH₃)—) and (—CH₂—CH(CH₃)—CH₂—).

In a further particularly preferred embodiment, the structural units of the formula (Si-amino) represent repeat units in the amino-functionalized silicone polymer, so that the silicone polymer comprises several structural units of the formula (Si-amino).

In the following, particularly well-suited amino-functionalized silicone polymers with at least one secondary amino group are listed.

Colorings with the best wash-fastness could be obtained if at least one agent containing at least one amino-functionalized silicone polymer comprising structural units of formula (Si—I) and formula (Si-II) was applied to the keratinous material during the coloring:

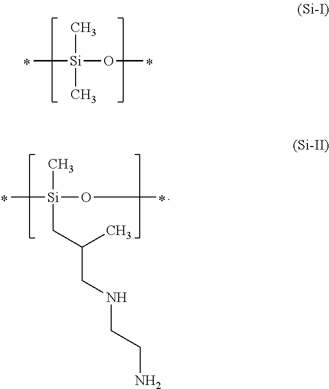

In a further explicitly very particularly preferred embodiment, a method according to the invention is characterized in that the coloring agent (F) contains at least one amino-functionalized silicone polymer (f1) which comprises structural units of formula (Si—I) and formula (Si-II):

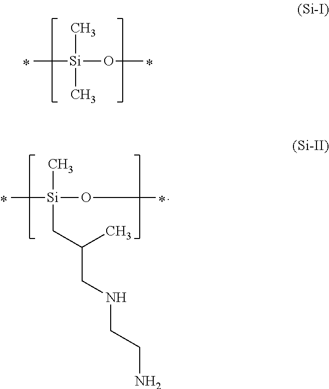

A corresponding amino-functionalized silicone polymer with the structural units (Si—I) and (Si-II) is, for example, the commercial product DC 2-8566 or Dowsil 2-8566 Amino Fluid which is sold commercially by Dow Chemical Company and which bears the designation, “Siloxanes and Silicones, 3-[(2-Aminoethyl)amino]-2-methylpropyl Me, Di-Me-Siloxane” and the CAS number 106842-44-8. A further particularly preferred commercial product is Dowsil AP-8568 Amino Fluid, which is likewise sold commercially by Dow Chemical Company.

In the context of a further embodiment, the coloring can also be carried out by applying a coloring agent (F) which contains at least one amino-functional silicone polymer of formula (Si—III),

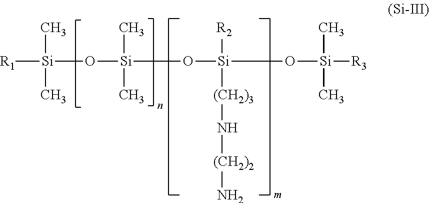

where
m and n denote numbers that are selected such that the sum (n+m) is in a range of 1 to 1,000,
n is a number in a range of 0 to 999, and m is a number in a range of 1 to 1,000,
R1, R2, and R3, which are identical or different, denote a hydroxyl group or a C₁-4 alkoxy group,
at least one of the R1 through R3 groups denotes a hydroxyl group.

Further suitable methods are characterized by the of a coloring agent to the hair, wherein the coloring agent contains at least amino-functional silicone polymer of formula (Si—IV),

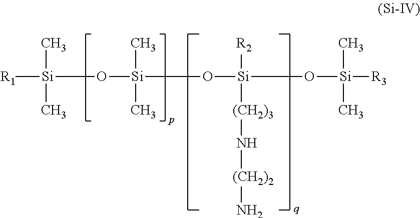

where
p and q denote numbers that are selected such that the sum (p+q) is in a range of 1 to 1,000,
p is a number in a range of 0 to 999, and q is a number in a range of 1 to 1,000, and
R1 and R2, which are different, denote a hydroxyl group or a C1-4 alkoxy group, at least one of the groups R1 through R2 denotes a hydroxyl group.

The silicones of the formulas (Si—III) and (Si—IV) differ by the moiety on the Si atom that carries the nitrogen-containing group: In formula (Si—III), R2 denotes a hydroxyl group or a C1-4 alkoxy group, whereas the group in formula (Si—IV) is a methyl group. The individual Si moieties, which are labeled with the indices m and n or p and q, need not be present as blocks; instead, the individual units can also be distributed randomly, i.e., in the formulas (Si—III) and (Si—IV), each R1-Si(CH₃)₂ group is not necessarily bound to a —[O—Si(CH₃)₂] moiety.

Methods according to the invention in which a coloring agent containing at least one amino-functional silicone polymer of formula of formula (Si—V) is applied to the hair have also proven to be particularly effective with regard to the generation of intense color results:

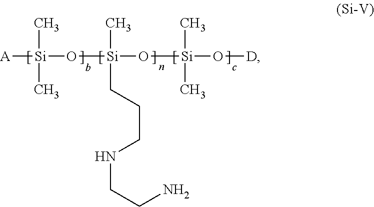

where
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
b, n, and c stand for integers between 0 and 1,000,
with the proviso that
   n >0 and b+c >0
   at least one of the conditions A=—OH or D=—H is met.

In the aforementioned formula (Si—V), the individual siloxane units having the indices b, c, and n are randomly distributed, i.e., they are not necessarily block copolymers.

The applied coloring agent can also contain one or more different amino-functionalized silicone polymers which are described by the formula (Si—VI):

M(RₐQ_{b}SiO_{(4-a-b)/2)x}(R_{c}SiO_{(4-c)2)y}M (Si—VI).

In the above formula, R is a hydrocarbon or a hydrocarbon group having 1 to approximately 6 carbon atoms, Q is a polar group of general formula —R¹HZ, in which R¹ is a bivalent linking group bonded to hydrogen and the group Z, composed of carbon and hydrogen atoms, carbon, hydrogen, and oxygen atoms, or carbon, hydrogen, and nitrogen atoms, and Z is an organic, amino-functional group containing at least one amino-functional group; “a” assumes values in a range of approximately 0 to approximately 2, “b” assumes values in a range of approximately 1 to approximately 3, “a”+“b” is less than or equal to 3, and “c” is a number in a range of approximately 1 to approximately 3, and x is a number in a range of 1 to approximately 2,000, preferably approximately 3 to approximately 50, and most preferably approximately 3 to approximately 25, and y is a number in a range of approximately 20 to approximately 10,000, preferably approximately 125 to approximately 10,000, and most preferably approximately 150 to approximately 1,000, and M is a suitable silicone end group as is known in the prior art, and preferably trimethylsiloxy. Non-limiting examples of the groups represented by R include alkyl groups, such as methyl, ethyl, propyl, isopropyl, isopropyl, butyl, isobutyl, amyl, isoamyl, hexyl, isohexyl, and the like; alkenyl groups such as vinyl, halovinyl, alkylvinyl, allyl, haloallyl, and alkylallyl; cycloalkyl groups such as cyclobutyl, cyclopentyl, cyclohexyl, and the like; phenyl groups; benzyl groups; halohydrocarbon groups such as 3-chloropropyl, 4-bromobutyl, 3,3,3-trifluoropropyl, chlorocyclohexyl, bromophenyl, chlorophenyl, and the like; and sulfur-containing groups such as mercaptoethyl, mercaptopropyl, mercaptohexyl, mercaptophenyl, and the like; R is preferably an alkyl group containing 1 to approximately 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃ CC(O)OCH₂CH₂—, —C₆H₄C₆H₄—, —C₆H₄CH₂C₆H₄—; and —(CH₂)₃C(O)SCH₂CH₂—.

Z is an organic, amino-functional group containing at least one functional amino group. A possible formula for Z is NH(CH₂)_{z}NH₂, where z is 1 or more. Another possible formula for Z is —NH(CH₂)z(CH₂)_{zz}NH, in which both z and zz are independently 1 or more, this structure comprising diamino ring structures, such as piperazinyl. Z is most preferably a —NHCH₂CH₂NH₂ functional group. Another possible formula for Z is N(CH2)z(CH2)_{zz}NX₂ or —NX₂, where each X of X₂ is selected independently from the group consisting of hydrogen and alkyl groups having 1 to 12 carbon atoms, and zz is 0.

Q is most preferably a polar, amino-functional group of formula —CH₂CH₂CH₂NHCH₂CH₂NH₂. In the formulas, “a” assumes values in the range of about 0 to about 2, “b” assumes values in the range of about 2 to about 3, “a”+“b” is less than or equal to 3, and “c” is a number in the range of about 1 to about 3. The molar ratio of the RₐQ_{b} SiO_{(4-a-b)/2} units to the R_{c}SiO_{(4-c)/2} units lies within a range of about 1:2 to 1:65, preferably about 1:5 to about 1:65, and most preferably about 1:15 to about 1:20. If one or more silicones of the above formula are used, then the various variable substituents in the above formula can be different in the various silicone components, present in the silicone mixture.

In the context of a further embodiment, a method according to the invention is characterized by the application of a coloring agent to the hair, wherein the coloring agent is an amino-functional silicone polymer of formula (Si—VII)

R′ₐG₃₋ₐ-Si(OSiG₂)ₙ-(OSiG_{b}R′_{2-b})ₘ—O—SiG₃₋ₐ-R′ₐ (Si—VII),
in which:
   G is —H, a phenyl group, —OH, —O—CH₃, —CH₃, —O—CH₂CH₃, —CH₂CH₃, —O—CH₂CH₂CH₃,—CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, or —C(CH₃)₃;
   a represents a number between 0 and 3, and in particular 0;
   b represents a number between 0 and 1, and in particular 1;
   m and n are numbers whose sum (m+n) is between 1 and 2,000, and preferably between 50 and 150, n preferably assuming values of 0 to 1,999 and in particular from 49 to 149, and m preferably assuming values of 1 to 2,000, and in particular from 1 to 10;
   R′ is a monovalent functional group selected from:
   Q-N(R″)—CH₂—CH₂—N(R″)₂
   -Q-N(R″)₂
   -Q-N⁺(R″)₃A⁻
   -Q-N⁺H(R″)₂ A⁻
   -Q-N⁺H₂(R″)A⁻
   -Q-N(R″)—CH₂—CH₂—N⁺R″H₂A⁻,
each Q representing a chemical bond, —CH₂—, —CH₂—CH₂—, —CH₂CH₂CH₂—, —C(CH₃)₂—, —CH₂CH₂CH₂CH₂—, —CH₂C(CH₃)₂—, or —CH(CH₃)CH₂CH₂—,
R″ representing identical or different functional groups from the group —H, -phenyl, -benzyl, —CH₂—CH(CH₃)Ph, from the C₁₋₂₀ alkyl groups, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A representing an anion preferably selected from chloride, bromide, iodide, or methosulfate.

In the context of a further embodiment, a method according to the invention is characterized by the application of a coloring agent to the hair, wherein the coloring agent contains at least one amino-functional silicone polymer of formula (Si-VIIa),
in which m and n are numbers of which the sum (m+n) is between 1 and 2,000, and preferably between 50 and 150, n preferably assuming values of 0 to 1,999 and in particular from 49 to 149, and m preferably assuming values of 1 to 2,000, and in particular from 1 to 10.

These silicones are designated as trimethylsilylamodimethicones in accordance with the INCI Declaration.

In the context of a further preferred embodiment, a method according to the invention is characterized by the application of a coloring agent to the hair, wherein the coloring agent contains at least one amino-functional silicone polymer of formula (Si-VIIb)
in which R denotes —OH, —O—CH₃, or a —CH₃ group and m, n1 and n2 are numbers whose sum (m+n1+n2) amounts to between 1 and 2,000, and preferably between 50 and 150, the sum (n1+n2) preferably assuming values from 0 to 1,999 and in particular from 49 to 149, and m preferably assuming values from 1 to 2,000, and in particular from 1 to 10.

According to the INCI Declaration, these amino-functionalized silicone polymers are referred to as amodimethicones.

Irrespective of which amino-functional silicones are used, coloring agents according to the invention that contain an amino-functional silicone whose amine value is above 0.25 meq/g, preferably above 0.3 meq/g, and in particular above 0.4 meq/g are preferred. The amine value here represents the milliequivalents of amine per gram of the amino-functional silicone. Said value can be determined by titration and may also be given in the unit, mg KOH/g.

Furthermore, coloring agents which contained a specific 4-morpholinomethyl-substituted silicone polymer are also suitable for use in the method according to the invention. This amino-functionalized silicone polymer comprises structural units of formulas (Si—VIII) and of the formula (Si—IX):

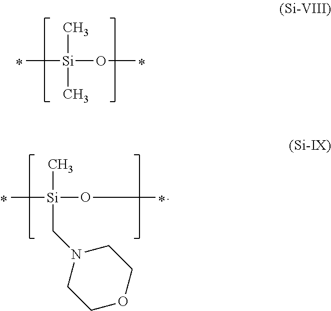

Corresponding 4-morpholinomethyl-substituted silicone polymers are described below.

A corresponding amino-functionalized silicone polymer is known under the name, amodimethicone/morpholinomethyl silsesquioxane copolymer, and is commercially available in the form of the raw material, Belsil ADM 8301 E from Wacker.

For example, a silicone which has structural units of formulas (Si—VIII), (Si—IX), and (Si—X) can be used as 4-morpholinomethyl-substituted silicone:

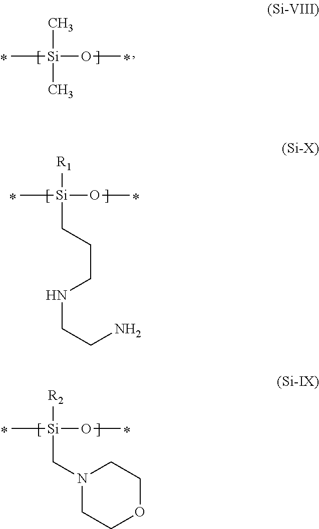

in which
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.

Particularly preferred coloring agents contain at least one 4-morpholinomethyl-substituted silicone of formula

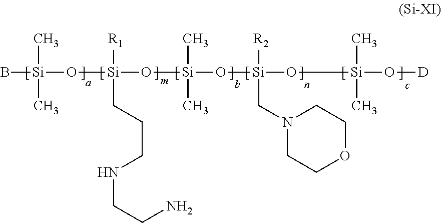

where
R1 represents —CH₃, —OH, —OCH₃, —O—CH₂CH₃, —O—CH₂CH₂CH₃, or —O—CH(CH₃)₂,
R2 represents —CH₃, —OH, or —OCH₃,
B represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
a, b, and c represent, independently of one another, integers between 0 and 1,000, with the proviso that a +b+c >0,
m and n represent, independently of one another, integers between 1 and 1,000,
with the proviso that
   at least one of the conditions B=—OH or D=—H is met,
   the units a, b, c, m, and n are distributed randomly or in blocks in the molecule.

Structural formula (Si—XI) is intended to indicate that the siloxane groups n and m do not necessarily have to be directly bonded to an end group B or D. Instead, in preferred formulas (Si—VI), a>0 or b>0 and, in particularly preferred formulas (Si—VI), a>0 and c>0; i.e., the terminal group B or D is preferably bonded to a dimethylsiloxy group. In formula (Si—VI) as well, the siloxane units a, b, c, m, and n are preferably distributed randomly.

The silicones represented by formula (Si—VI) and used according to the invention can be trimethylsilyl-terminated (D or B═—Si(CH₃)₃), but they may also be dimethylsilylhydroxy-terminated at both ends or dimethylsilylhydroxy- and dimethylsilylmethoxy-terminated at one end. Within the context of the present invention, silicones which are particularly preferably used are selected from silicones in which:
B═—O—Si(CH₃)₂OH and D=—Si(CH₃)₃
B═—O—Si(CH₃)₂OH and D=—Si(CH₃)₂OH
B═—O—Si(CH₃)₂OH and D=—Si(CH₃)₂OCH₃
B═—O—Si(CH₃)₃ and D=—Si(CH₃)₂OH
B═—O—Si(CH₃)₂OCH₃ and D=—Si(CH₃)₂OH.
These silicones lead to enormous improvements in the hair properties of hair treated with the agents according to the invention, and to greatly improved protection during oxidative treatment.

The coloring agents used in the coloring step can contain one or more amino-functionalized silicone polymers, for example, in a total amount of 0.1 to 8.0 wt %, preferably 0.2 to 5.0 wt %, more preferably 0.3 to 3.0 wt % and very particularly preferably 0.4 to 2.5 wt %. Here, the indicated amounts relate to the total amount of all aminosilicones used, which is set in relation to the total weight of the coloring agent.

In the context of another particularly preferred embodiment, a method according to the present invention is characterized in that the coloring agent (F)—relative to the total weight of the coloring agent (F)—contains one or more amino-functionalized silicone polymers (f1) in a total amount of 0.1 to 20 wt %, preferably from 0.2 to 10 wt %, more preferably from 0.3 to 5 wt %, still more preferably from 0.4 to 3.5 wt %, and very particularly preferably from 0.5 to 2.0 wt %.

### Pigments (f2) in the Coloring Agent (F)

As a second essential component, the coloring agent (F) applied in step (1) of the method according to the invention contains at least one pigment (f2).

Pigments in the sense of the present invention are understood to mean coloring compounds which have a solubility of less than 0.5 g/L, preferably of less than 0.1 g/L, and even more preferably of less than 0.05 g/L, at 25° C. in water. The water solubility can be determined, for example, by means of the method described below: 0.5 g of the pigment is weighed into a beaker. A stir bar is added. Then one liter of distilled water is added. This mixture is heated to 25° C. while stirring with a magnetic stirrer for one hour. If still undissolved components of the pigment are visible in the mixture after this period, the solubility of the pigment is below 0.5 g/L. If the pigment-water mixture cannot be visually assessed due to the high intensity of the pigment that may be finely dispersed, the mixture is filtered. If a portion of undissolved pigments remains on the filter paper, the solubility of the pigment is below 0.5 g/L.

Suitable dye pigments may be of inorganic and/or organic origin. In a preferred embodiment, a method according to the invention is characterized in that the post-treatment agent is applied to keratin material which has been dyed by application of at least one inorganic and/or organic pigment.

Preferred color pigments are selected from synthetic or natural inorganic pigments. Inorganic color pigments of natural origin can be produced, for example, from chalk, ocher, umbra, green soil, burnt Sienna, or graphite. Furthermore, black pigments such as, for example, iron oxide black, chromatic pigments such as, for example, ultramarine or iron oxide red, and also fluorescent or phosphorescent pigments, can be used as inorganic color pigments.

Colored metal oxides, hydroxides and oxide hydrates, mixed phase pigments, sulfur-containing silicates, silicates, metal sulfides, complex metal cyanides, metal sulfates, chromates, and/or molybdates are particularly suitable. Particularly preferred color pigments are black iron oxide (CI 77499), yellow iron oxide (CI 77492), red and brown iron oxide (CI77491), manganese violet (CI77742), ultramarine (sodium aluminum sulphosilicates, Cl 77007, Pigment Blue 29), chromium oxide hydrate (CI77289), Iron Blue (ferric ferrocyanide, C177510) and/or carmine (cochineal).

Color pigments which are likewise particularly preferred according to the invention are colored pearlescent pigments. These are usually based upon mica and may be coated with one or more metal oxides. Mica is a phyllosilicate. The most important representatives of these silicates are muscovite, phlogopite, paragonite, biotite, lepidolite, and margarite. In order to produce the pearlescing pigments in conjunction with metal oxides, mica, and primarily muscovite or phlogopite, is coated with a metal oxide.

As an alternative to natural mica, synthetic mica coated with one or more metal oxides(s) can also be used as a pearlescent pigment. Particularly preferred pearlescent pigments are based upon natural or synthetic mica and are coated with one or more of the aforementioned metal oxides. The color of the respective pigments can be varied by varying the layer thickness of the metal oxide(s).

In another preferred embodiment, a method according to the invention is characterized in that the coloring agent (F) contains at least one inorganic pigment (f2), wherein the inorganic pigment is preferably selected from the group consisting of colored metal oxides, metal hydroxides, metal oxide hydrates, silicates, metal sulfides, complex metal cyanides, metal sulphates, bronze pigments, and/or from mica-based colored pigments which are coated with at least one metal oxide and/or a metal oxychloride.

In another preferred embodiment, a method according to the invention is characterized in that the coloring agent (F) contains at least one pigment (f2) selected from mica-based colored pigments which are coated with one or more metal oxides from the group of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288), and/or iron blue (ferric ferrocyanide, CI 77510).

Examples of particularly suitable color pigments are commercially available, for example, under the trade names, Rona®, Colorona®, Xirona®, Dichrona®, and Timiron® from the company Merck, Ariabel® and Unipure® from the company Sensient, Prestige® from the company Eckart Cosmetic Colors, and Sunshine® from the company Sunstar.

Very particularly preferred color pigments with the trade name, Colorona®, are, for example:
Colorona Copper, Merck, MICA, CI 77491 (IRON OXIDES)
Colorona Passion Orange, Merck, Mica, CI 77491 (IRON OXIDES), Alumina
Colorona Patina Silver, Merck, MICA, CI77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES)
Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE
Colorona Blackstar Green, Merck, MICA, CI 77499 (IRON OXIDES)
Colorona Bordeaux, Merck, MICA, CI 77491 (IRON OXIDES)
Colorona Bronze, Merck, MICA, CI 77491 (IRON OXIDES)
Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA
Colorona Sienna, Merck, MICA, CI 77491 (IRON OXIDES)
Colorona Precious Gold, Merck, Mica, CI 77891 (Titanium dioxide), Silica, CI 77491 (IRON OXIDES), Tin oxide
Colorona Sun Gold Sparkle MP 29, Merck, MICA, TITANIUM DIOXIDE, IRON OXIDES, MICA, CI 77891, CI 77491 (EU)
Colorona Mica Black, Merck, CI 77499 (Iron oxides), Mica, CI 77891 (Titanium dioxide)
Colorona Bright Gold, Merck, Mica, CI 77891 (Titanium dioxide), CI 77491(Iron oxides)
Colorona Blackstar Gold, Merck, MICA, CI 77499 (IRON OXIDES)

Additional particularly preferred color pigments with the trade name, Xirona®, are, for example:
Xirona Golden Sky, Merck, Silica, CI 77891 (Titanium Dioxide), Tin Oxide
Xirona Caribbean Blue, Merck, Mica, CI 77891 (Titanium Dioxide), Silica, Tin Oxide
Xirona Kiwi Rose, Merck, Silica, CI 77891 (Titanium Dioxide), Tin Oxide
Xirona Magic Mauve, Merck, Silica, CI 77891 (Titanium Dioxide), Tin Oxide.
In addition, particularly preferred color pigments with the trade name, Unipure®, are, for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica

In the context of a further embodiment, the applied coloring agent can also contain one or more organic pigments.

The organic pigments according to the invention are correspondingly insoluble, organic dyes or color lacquers, which may be selected, for example, from the group of nitroso, nitro, azo, xanthene, anthraquinone, isoindolinone, isoindoline, quinacridone, perinone, perylene, diketopyrrolopyrrole, indigo, thioindigo, dioxazine, and/or triarylmethane compounds.

Particularly well-suited organic pigments can for example include carmine, quinacridone, phthalocyanine, sorghum, blue pigments with the Color Index numbers, CI 42090, CI 69800, CI 69825, CI 73000, CI 74100, or CI 74160, yellow pigments with the Color Index numbers, CI 11680, CI 11710, CI 15985, CI 19140, CI 20040, CI 21100, CI 21108, CI 47000, or CI 47005, green pigments with the Color Index numbers, CI 61565, CI 61570, or CI 74260, orange pigments with the Color Index numbers, CI 11725, CI 15510, CI 45370, or CI 71105, and red pigments with the Color Index numbers, CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, and/or CI 75470.

In another particularly preferred embodiment, a method according to the invention is characterized in that the coloring agent (F) contains at least one organic pigment (f2), wherein the organic pigment is preferably selected from the group consisting of carmine, quinacridone, phthalocyanine, sorghum, blue pigments with the Color Index numbers, CI 42090, CI 69800, CI 69825, CI 73000, CI 74100, or CI 74160, yellow pigments with the Color Index numbers, CI 11680, CI 11710, CI 15985, CI 19140, CI 20040, CI 21100, CI 21108, CI 47000, or CI 47005, green pigments with the Color Index numbers, CI 61565, CI 61570, or CI 74260, orange pigments with the Color Index numbers, CI 11725, CI 15510, CI 45370, or CI 71105, and red pigments with the Color Index numbers, CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, and/or CI 75470.

The organic pigment can also be a color lacquer. The term color lacquer in the sense of the invention is understood to mean particles which comprise a layer of absorbed dyes, with the unit consisting of particles and dye being insoluble under the above-mentioned conditions. The particles may, for example, be inorganic substrates which may be aluminum, silica, calcium borosilicate, calcium aluminum borosilicate, or aluminum.

For example, the alizarin color lacquer can be used as the color lacquer.

For the coloring of the hair, pigments with a specific shaping may also be used. For example, a pigment based upon a lamellar and/or lenticular substrate platelet may be used. Furthermore, the coloring is also possible based upon a substrate platelet which comprises a vacuum-metalized pigment.

In the context of another preferred embodiment, a method according to the invention is characterized in that the coloring agent (F) contains at least one pigment (f2) selected from the group of pigments based upon a lamellar substrate platelet, pigments based upon a lenticular substrate platelet, and vacuum-metalized pigments.

The substrate platelets of this type have an average thickness of at most 50 nm, preferably less than 30 nm, and particularly preferably at most 25 nm—for example, at most 20 nm. The average thickness of the substrate platelets is at least 1 nm, preferably at least 2.5 nm, and particularly preferably at least 5 nm—for example, at least 10 nm. Preferred ranges for the thickness of the substrate platelets are 2.5 to 50 nm, 5 to 50 nm, 10 to 50 nm; 2.5 to 30 nm, 5 to 30 nm, 10 to 30 nm; 2.5 to 25 nm, 5 to 25 nm, 10 to 25 nm, 2.5 to 20 nm, 5 to 20 nm, and 10 to 20 nm. Preferably, each substrate platelet has as uniform a thickness as possible. Due to the small thickness of the substrate platelets, the pigment has a particularly high covering power.

The substrate platelets have a preferably monolithic structure. Monolithic in this context means consisting of a single self-contained unit without fractures, stratifications, or inclusions, although structural changes may, however, occur within the substrate platelets. The substrate platelets are preferably composed homogeneously, i.e., there is no concentration gradient within the platelets. In particular, the substrate platelets are not constructed in layers and have no particles distributed therein.

The size of the substrate platelet can be matched to the respective application, and in particular to the desired effect on the keratin material. In general, the substrate platelets have an average largest diameter of approximately 2 to 200 μm, and in particular approximately 5 to 100 μm.

In a preferred embodiment, the form factor (aspect ratio), expressed by the ratio of the average size to the average thickness, is at least 80, preferably at least 200, more preferably at least 500, and particularly preferably more than 750. In this case, the average size of the uncoated substrate platelets is understood to mean the d50 value of the uncoated substrate platelets. Unless stated otherwise, the d50 value was determined using a device of the Sympatec Helos type, having Quixel wet dispersion. To prepare the sample, the sample to be investigated was pre-dispersed in isopropanol for a period of 3 minutes.

The substrate platelets may be constructed from any material that can be made into the form of a platelet. They can be of natural origin, but can also be produced synthetically. Materials from which the substrate platelets can be constructed are, for example, metals and metal alloys, metal oxides, and preferably aluminum oxide, inorganic compounds, and minerals such as mica and (semi-)precious stones, as well as plastics. Preferably, the substrate platelets are made of metal (alloy)s.

Any metal suitable for metallic luster pigments is suitable as the metal. Such metals are, inter alia, iron and steel, and all air-resistant and water-resistant (semi) metals such as, for example, platinum, zinc, chromium, molybdenum, and silicon, as well as alloys thereof such as aluminum bronzes and brass. Preferred metals are aluminum, copper, silver, and gold. Preferred substrate platelets are aluminum platelets and brass platelets, wherein substrate platelets made of aluminum are particularly preferred.

Lamellar substrate platelets are characterized by an irregularly structured edge, and are also referred to as “cornflakes” due to their appearance. Due to their irregular structure, pigments based upon lamellar substrate platelets produce a high fraction of scattered light. In addition, the pigments based upon lamellar substrate platelets do not completely cover the existing color of a keratin material, and, for example, effects can be achieved analogous to a natural graying.

Lenticular (=lens-shaped) substrate platelets have a substantially regular round edge and are also referred to as “silver dollars” due to their appearance. Due to their regular structure, the fraction of the reflected light predominates in the case of pigments based upon lenticular substrate platelets.

Vacuum-metalized pigments (VMP) can be obtained, for example, by releasing metals, metal alloys, or metal oxides from correspondingly coated films. These are characterized by a particularly small thickness of the substrate platelets in the range from 5 to 50 nm and by a particularly smooth surface having increased reflectivity. Substrate platelets which comprise a pigment metalized in a vacuum are also referred to as VMP substrate platelets in the context of this application. VMP substrate platelets of aluminum can be obtained, for example, by releasing aluminum from metalized films.

The substrate platelets made of metal or metal alloy can be passivated—for example, by anodizing (oxide layer) or chromatizing.

Uncoated lamellar, lenticular, and/or VPM substrate platelets, and in particular those made of metal or metal alloy, reflect the incident light to a high degree and produce a light-dark flop. These have proven to be particularly preferred for use in the coloring agent.

Suitable pigments based upon a lamellar substrate platelet include, for example, the pigments of the VISIONAIRE series from Eckart.

Pigments based upon a lenticular substrate platelet are available, for example, under the name, Alegrace® Gorgeous, from the company Schlenk Metallic Pigments GmbH.

Pigments based upon a substrate platelet, which comprises a vacuum-metalized pigment, are available, for example, under the name, Alegrace® Marvelous or Alegrace® Aurous from the company Schlenk Metallic Pigments GmbH.

Owing to their excellent light and temperature resistance, the use of the aforementioned pigments in the method according to the invention is very particularly preferred. It is further preferred if the pigments used have a certain particle size. It is therefore advantageous according to the invention if the at least one pigment has a mean particle size D₅₀ of 1.0 to 50 μm, preferably 5.0 to 45 μm, preferably 10 to 40 μm, and in particular 14 to 30 μm. The mean particle size D₅₀ can be determined, for example, using dynamic light scattering (DLS).

The pigments (f2) are preferably used in specific quantity ranges in the coloring agent (F). The coloring agent used in the method according to the invention for coloring may contain one or more pigments, for example in a total amount of 0.01 to 10.0 wt %, preferably 0.1 to 5.0 wt %, more preferably 0.2 to 2.5 wt %, and very particularly preferably 0.25 to 1.5 wt %. Here, the indicated amounts relate to the total amount of all pigments used, which is set in relation to the total weight of the coloring agent.

In another very particularly preferred embodiment, a method according to the invention is characterized in that the coloring agent (F) contains—relative to the total weight of the coloring agent (F)—one or more pigments (f2) in a total amount of 0.01 to 10.0 wt %, preferably 0.1 to 5.0 wt %, more preferably 0.2 to 2.5 wt %, and very particularly preferably 0.25 to 1.5 wt %.

### Water Content in the Coloring Agent (F)

The previously described coloring agent (F) is a ready-to-use agent which is applied to the hair. This ready-to-use agent preferably possesses a low to medium water content. It has been found that particularly those coloring agents are well-suited which contain—relative to the total weight of the agent—0.1 to 50.0 wt %, preferably 0.5 to 35.0 wt %, more preferably 1.0 to 20.0 wt %, and particularly preferably 1.5 to 15.0 wt % water.

In another explicitly very particularly preferred embodiment, a method is characterized in that the coloring agent (F) contains—relative to the total weight of the coloring agent (F)—0.1 to 50.0 wt %, preferably 0.5 to 35.0 wt %, more preferably 1.0 to 20.0 wt %, and particularly preferably 1.5 to 15.0 wt % water.

### Cosmetic Carrier of Coloring Agent (F)

Due to the previously described water content of the coloring agent, which is preferably in the medium to low range, the main component of the cosmetic carrier in which the components (f1) and (f2) of the coloring agent are present is preferably non-aqueous. The cosmetic carrier is preferably a solvent and/or a polyethylene glycol.

Suitable solvents that can be used are, for example, solvents from the group consisting of 1,2-propylene glycol, 1,3-propylene glycol, 1,2-butylene glycol, dipropylene glycol, ethanol, isopropanol, diethylene glycol monoethyl ether, glycerol, phenoxyethanol, and benzyl alcohol. The use of 1,2-propylene glycol is very particularly preferred.

A very particularly preferred low molecular weight polyethylene glycol is PEG-8, for example. PEG-8 comprises, on average, 8 ethylene glycol units (x1=8), has an average molecular weight of 400 g/mol, and bears the CAS number 25322-68-3. PEG-8 is alternatively also referred to as PEG 400 and is commercially available, for example, from APS.

Additional well-suited low molecular weight polyethylene glycols are, for example, PEG-6, PEG-7, PEG-9, and PEG-10.

Another well-suited polyethylene glycol is PEG-32, for example. PEG-32 comprises 32 ethylene glycol units (x1=32), has a mean molar mass of 1,500 g/mol, and bears the CAS number 25322-68-3. PEG-32 is alternatively also referred to as PEG 1500 and can, for example, be purchased commercially from Clariant.

A very particularly well-suited polyethylene glycol with an average molecular weight is, for example, PEG 6000, which can be obtained commercially from the National Starch company (China). The molecular weight of PEG 6000 is 6,000 to 7,500 g/mol, corresponding to an x3 value of 136 to 171.

### Further Optional Ingredients in the Coloring Agent (F)

In addition to the previously described components that are essential to the invention and preferred components, the coloring agent can also contain other optional ingredients, such as, for example, surfactants, anionic, non-ionic, zwitterionic, and/or cationic polymers; structurants such as glucose, maleic acid and lactic acid, hair-conditioning compounds such as phospholipids, e.g., lecithin and cephalins; perfume oils, dimethyl isosorbide, and cyclodextrins; fiber structure-improving agents, and in particular mono-, di-, and oligosaccharides, e.g., glucose, galactose, fructose, and lactose; dyes for coloring the product; anti-dandruff active ingredients such as piroctone olamine, zinc omadine, and climbazole; amino acids and oligopeptides; animal and/or vegetable-based protein hydrolysates, as well as in the form of their fatty acid condensation products or optionally anionically- or cationically-modified derivatives; vegetable oils; light stabilizers and UV blockers; active ingredients such as panthenol, pantothenic acid, pantolactone, allantoin, pyrrolidinone carboxylic acids and their salts, and bisabolol; polyphenols, and in particular hydroxycinnamic acids, 6,7-dihydroxycoumarins, hydroxybenzoic acids, catechins, tannins, leucoanthocyanidins, anthocyanidins, flavanones, flavones, and flavonols; ceramides or pseudoceramides; vitamins, provitamins, and vitamin precursors; plant extracts; fats and waxes such as fatty alcohols, beeswax, montan wax, and kerosenes; swelling and penetrating agents such as glycerol, propylene glycol monoethyl ether, carbonates, hydrogen carbonates, guanidines, ureas, and primary, secondary, and tertiary phosphates; opacifiers such as latex, styrene/PVP, and styrene/acrylamide copolymers; pearlescent agents such as ethylene glycol mono- and distearate as well as PEG-3-distearate; and propellants such as propane-butane mixtures, N₂O, dimethyl ether, CO₂, and air.

The selection of these additional substances is made by the person skilled in the art according to the desired properties of the agents. With respect to other optional components and the employed amounts of said components, reference is made expressly to relevant manuals known to the person skilled in the art. The additional active ingredients and auxiliaries are used in the preparations according to the invention preferably always in amounts of 0.0001 to 25 wt %, and in particular of 0.0005 to 15 wt %, relative to the total weight of the particular agent.

### Applying the Coloring Agent (F)

The previously described coloring agent (F) is first applied to the hair, wherein the hair can be moistened or dry. The coloring agent is applied, for example, with the gloved hand or with the aid of a brush, an applicator, or an application tool.

After application, the coloring agent is preferably allowed to act upon the hair. A great advantage of the coloring system according to the invention is that an intensive color result can be achieved even in very short periods after short exposure times. For this reason, it is advantageous for the coloring agent to remain on the keratin material after its application only for comparatively short periods from 30 seconds to 15 minutes, preferably from 30 seconds to 10 minutes, and particularly preferably from 1 to 5 minutes.

Subsequently, the coloring agent is preferably rinsed or washed out of the hair, wherein the washing out preferably takes place with water without the aid of a shampoo and without using another cosmetic or surfactant formulation.

In the context of a further preferred embodiment, a method according to the invention is characterized by the following steps in the stated sequence:
(1a) applying the coloring agent to the hair,
(1b) allowing the coloring agent to act upon the hair for a period of 1 to 30 minutes, and preferably 1 to 5 minutes,
(1c) rinsing out the coloring agent with water, and
(2) irradiating the hair for a period of 1 to 60 minutes with UV/VIS radiation in the wavelength range of 200 to 800 nm.

The hair can in principle be irradiated at different points in time. The coloring and the irradiation do not necessarily have to be carried out directly one after the other, and a period of several hours can pass between the coloring step (1) (or the coloring steps (1a), (1b), and (1c)) and the irradiation (2). It is thus possible, for example, to first color and dry the hair, and then to carry out the irradiation on the dried hair.

However, a particularly good curing of the film comprising aminosilicone and pigment could be obtained if the irradiation (2) directly follows the coloring step (1). The greatest improvement in wash-fastness was obtained when the still-moist hair was irradiated with an artificial radiation source directly after the coloring.

Therefore, a method is explicitly very particularly preferred which is characterized by the following steps in the stated sequence:
(1a) applying the coloring agent to the hair,
(1b) allowing the coloring agent to act upon the hair for a period of 1 to 30 minutes, and preferably 1 to 5 minutes,
(1c) rinsing out the coloring agent with water,
(2) irradiating the hair, and preferably the still-moist hair, for 1 to 60 minutes with an artificial light source which emits UV/VIS radiation in the wavelength range of 200 to 800 nm,
   wherein a period of a maximum of 72 hours, preferably a maximum of 48 hours, more preferably a maximum of 24 hours, even more preferably a maximum of 2 hours, and most preferably a maximum of 15 minutes passes between steps (1c) and (2).

Still-moist hair is understood here to mean hair from which the coloring agent was rinsed with water and which was then rubbed dry with a towel. The person skilled in the art will also call hair with this degree of moisture (still moist, but no longer dripping wet) towel-dry or towel-moist hair.

### Irradiating the Hair

In step (2) of the method according to the invention, the hair colored in step (1) is irradiated with UV/VIS radiation in the wavelength range of 200 to 800 nm for a period of 1 to 60 minutes. Irradiation in the sense of the invention is to be understood as an irradiation with an artificial light source which emits the light in the wavelength range of 200 to 800 nm. A person with colored hair spending time under daylight conditions is therefore not to be understood as irradiation in the sense of the invention.

In other words, the first subject matter of the invention is a method for coloring human hair, comprising
(1) applying a coloring agent (F) to the hair, which includes
(f1) at least one amino-functionalized silicone polymer, and
(f2) at least one pigment, and
(2) irradiating the hair treated in step (1) with an artificial light source for a period of 1 to 60 minutes with UV/VIS radiation in the wavelength range of 200 to 800 nm.

Or, in other words, the first subject matter of the invention is a method for coloring human hair, comprising
(1) applying a coloring agent (F) to the hair, which includes
(f1) at least one amino-functionalized silicone polymer, and
(f2) at least one pigment, and
(2) irradiating the hair treated in step (1) for a period of 1 to 60 minutes with an artificial light source, wherein the artificial light source emits UV/VIS radiation in the wavelength range of 200 to 800 nm.

UV/VIS radiation comprises the UV range (ultraviolet range) and the VIS range (visible range) as electromagnetic radiation in the wavelength range of 200 nm to 800 nm.

UV radiation (ultraviolet radiation) is electromagnetic waves having a wavelength of 380 to 10 nm or a frequency of approximately 790 THz to 30 PHz. The energy of a single light quantum is in the range of ca. 3.3 eV (380 nm) to ca. 124 eV (10 nm).

The light visible to the human eye is the part of the electromagnetic spectrum with wavelengths between about 380 and 780 nm. Light having a wavelength of 800 nm is still barely visible.

In the method according to the invention, a coloring agent is applied to the hair to be treated and optionally rinsed out, and the hair is subsequently irradiated with UV light and/or visible light. Since the head of the person who is being treated is exposed to UV light, the usual precautions in handling ultraviolet radiation are to be observed. In particular, an excessively long period of irradiation should be avoided. Here, methods according to the invention are preferred in which the treated hair is irradiated, after application of the agent, for 1 to 60 minutes, preferably 2 to 50 minutes, in particular 5 to 40 minutes, and in particular 10 to 30 minutes.

In the context of a further preferred embodiment, a method according to the invention is characterized by (2) irradiating the hair for a period of 2 to 50 minutes, in particular 5 to 40 minutes, and in particular 10 to 30 with an artificial light source which emits electromagnetic radiation having wavelengths in the range of 200 to 800 nm.

Without being limited to this theory, it is assumed that the irradiation of the colored hair starts chemical reactions which alter the nature and/or chemical structure of the film of aminosilicones and pigments. An observation here was that particularly rapid and effective curing of the film takes place in particular due to the high-energy components of the electromagnetic beams. When curing this film, its solubility is reduced, and the wash resistance is increased in this way.

Even if a certain curing already takes place due to the visible parts of the light, this effect is nevertheless particularly pronounced when electromagnetic radiation having shorter wavelengths is used. For this reason, it has been found to be particularly preferred if the colored hair is irradiated with UV-A radiation having a wavelength of 400 nm-320 nm, UV-B radiation having a wavelength of 320 nm-280 nm, and/or UV-C radiation having a wavelength of 280 nm-200 nm.

In the context of a further particularly preferred embodiment, a method according to the invention is characterized by (2) irradiating the hair with UV-A radiation having a wavelength of 400 nm-320 nm, UV-B radiation having a wavelength of 320 nm-280 nm, and/or UV-C radiation having a wavelength of 280 nm-200 nm.

In the context of a further preferred embodiment, a method according to the invention is characterized by
(2) irradiating the hair for a period of 2 to 50 minutes, in particular 5 to 40 minutes, and in particular 10 to 30 with an artificial light source, which emits UV-A radiation having a wavelength of 400 nm-320 nm, UV-B radiation having a wavelength of 320 nm-280 nm, and/or UV-C radiation having a wavelength of 280 nm-200 nm.

Various light sources are suitable for generating electromagnetic radiation, and in particular for generating the previously described UV-A, UV-B, and UV-C radiation. For example, gas discharge tubes, gas discharge lamps, light-emitting diodes (LED's), pulsed xenon lamps, and/or an excimer lamps can thus be used to generate electromagnetic radiation in the desired wavelength range.

In the context of a further preferred embodiment, a method according to the invention is characterized in that the irradiation (2) is carried out with the aid of a gas discharge tube, a gas discharge lamp, a light-emitting diode, a pulsed xenon lamp, and/or an excimer lamp.

The gas discharge tube is an arrangement of cathode and anode within a gas-filled glass tube, in which a gas discharge with emission of light occurs when a design-specific minimum voltage is applied. Gas discharge lamps or gas discharge tubes consist of a usually tubular discharge vessel made of glass (low-pressure lamps), of quartz glass (high and ultra high-pressure lamps), or of aluminum oxide ceramic (high-pressure lamps). Two electrodes, between which an electric field is established and a gas discharge takes place, are located in the housing. The gas discharge lamps require a current limitation for operation, since otherwise the charge carrier density and the current rise rapidly due to the shock ionization, which, however, in the lamps leads to their destruction. The current limitation is achieved by a resistor (glow lamp), a choke, or an electronic ballast. The discharge vessel can be filled with a gas or gas mixture, but can also contain substances which become active by evaporation only at a later point in time. At room temperature, these gases in the vessel have a low pressure, which favors the ignition of the discharge by impact ionization and thus the generation of a plasma. The filling with gas mixtures determines the properties of the discharge or serves the sole purpose of providing enough heat to evaporate the substance intended for the actual plasma. Due to the resulting temperatures, a supply of the plasma-forming material located in the vessel is evaporated, and thus increases the pressure in the discharge space.

The plasma-forming substances are metals or vapors thereof (such as sodium or mercury), wherein, for ignition, noble gases are always also present, or pure noble gases (such as xenon, krypton, and neon) or mixtures of halogens and metals (halogen metal vapor lamps).

High-pressure gas discharge tubes or lamps are also referred to as HID lamps (high-intensity discharge). Their current and luminance densities are substantially higher than in low-pressure plasmas; the discharge works in the region of an arc or an arc discharge.

A particularly well-suited gas discharge lamp is, for example, the mercury vapor lamp. In addition to the mercury, which, due to the vapor pressure already being low at room temperature, is partially present in gaseous form, the mercury vapor lamp also contains a noble gas (usually argon) in order to facilitate ignition.

The low-pressure mercury vapor lamp is a lamp type widely used to generate UV-C radiation. The main emission in this case—often of more than 90 percent—is at a wavelength of 253.7 nm. In addition, other wavelengths are emitted in the ultraviolet, visible, and infrared range. The use of a low-pressure mercury vapor lamp for irradiating the colored hair in step (2) of the method according to the invention is very particularly preferred.

In the context of a further preferred embodiment, a method according to the invention is characterized in that the irradiation (2) is carried out using an artificial radiation source which converts at least 60% of its energy, preferably at least 70% of its energy, more preferably at least 80% of its energy, and particularly preferably at least 85% of its energy into UV-C radiation having a wavelength of 250 nm to 260 nm.

In the context of a further preferred embodiment, a method according to the invention is characterized in that the irradiation (2) is carried out using a low-pressure mercury vapor lamp which converts at least 60% of its energy, preferably at least 70% of its energy, more preferably at least 80% of its energy, and particularly preferably at least 85% of its energy into UV light having a wavelength of 250 nm to 260 nm, and in particular having a wavelength of 253.7 nm.

A very well-suited low-pressure mercury vapor discharge lamp is sold, for example, by the company Philips MSR under the name, “Philips TUV PL-S 9W 2P G23 disinfection.” The lamp emits UV-C radiation with a peak value of 253.7 nm (UV-C). The glass vessel of the lamp filters out the ozone-forming 185 nm region.

In contrast to low-pressure mercury lamps, high-pressure mercury lamps are not line emitters, but broadband UV-C emitters. This lamp type is likewise a very intense and well-suited radiation source.

The group of gas discharge tubes or gas discharge lamps also includes lighting tubes or fluorescent tubes, which are also referred to as cold cathode fluorescent lamps (CCFL for short). These are gas discharge tubes, between the electrodes of which a glow discharge ignites upon application of a high voltage, and the extended positive column of which glows in a colored manner depending upon the filling gas. The cathode is unheated and therefore hardly emits thermal electrons. The emission takes place by secondary electron emission of positive ions that are accelerated in the cathode case and collide with the cathode.

A particularly well-suited fluorescent tube can be commercially obtained from the company Sylvanio, for example, under the name, “Sylvania Blacklight F15 15W T8 BL368 G13 fluorescent tube.” This lamp emits UV radiation in the range of 350-400 nm with a maximum at 368 nm. This T8 tube has a total length of 437 mm and a diameter of 26 mm.

In the context of a further preferred embodiment, a method according to the invention is characterized in that the irradiation (2) is carried out using an artificial radiation source which converts at least 60% of its energy, preferably at least 70% of its energy, more preferably at least 80% of its energy, and particularly preferably at least 85% of its energy into UV-A radiation having a wavelength of 350 nm to 380 nm.

In the context of a further preferred embodiment, a method according to the invention is characterized in that the irradiation (2) is carried out using a fluorescent tube which converts at least 60% of its energy, preferably at least 70% of its energy, more preferably at least 80% of its energy, and particularly preferably at least 85% of its energy into UV-A radiation having a wavelength of 350 nm-380 nm, and in particular having a wavelength of 368 nm.

Other suitable radiation sources are, for example, pulsed xenon lamps, excimer lamps, and light-emitting diodes (LED's). Depending upon the lamp type, they can radiate very specific wavelengths in the UV-C range, or also additional wavelengths in the visible and infrared range can be emitted. By using so-called optical filters, the emitted wavelength range can be limited if required.

The pulsed xenon lamps are lamps which emit a short pulse with a broad spectrum which comprises the ultraviolet, visible, and infrared wavelength ranges. By corresponding filtering of the spectrum, these lamps emit primarily UV-C radiation.

The excimer lamp is also referred to as a far UV-C lamp. This is a quasi-monochromatic light source, which means that the excimer lamp emits UV-C radiation in narrow bands of a certain wavelength. In the case of a krypton-chlorine excimer lamp, this wavelength is, for example, 222 nm.

Following step (2) of the method according to the invention, further steps such as a post-treatment with a conditioner or a leave-on post-treatment agent may optionally also follow. A drying of the hair after step (2) is also in accord with the invention. Thus, the hair can, for example, also be dried, if it is not yet completely dry from the irradiation (2), with the aid of an external heat source such as a hairdryer.

### EXAMPLES

### 1. Formulations

The following formulation was produced (all information is in wt % unless indicated otherwise).

| Ready-to-use coloring agent (F) | F (wt %) |
|---|---|
| DOWSIL AP-8568 Amino Fluid (aminosilicone (f1)) | 0.75 |
| Hexamethyldisiloxane | 6.0 |
| Unipure Red LC 3079 (pigment (f2)) | 0.38 |
| Alegrace Marvelous D 12/77-1 Shiny Silver (10% dispersion | 2.30 |
| of the pigment) (pigment (f2)) | |
| Water | 10.0 |
| PPG-3 benzyl ether myristate | 1.4 |
| Ucon Fluid AP (PPG-14 butyl ether) | 1.0 |
| Phenoxyethanol | 0.8 |
| 4-hydroxyacetophenone | 0.2 |
| Polyethylene glycol (PEG 6000, molar weight 6,000 g/mol) | 10.0 |
| Polyethylene glycol (PEG 400, molar weight 400 g/mol) | up to 100 |

### 2. Application

Hair strands (Kerling, “Euronature hair white” type) were measured colorimetrically. The ready-to-use coloring agent F was then applied to the hair strands (liquor ratio: 1 g of agent per 1 g of hair strand) and allowed to act for three minutes. Subsequently, the hair strands were washed thoroughly (1 minute) with water. The hair strands were then dried with a towel.

The still-moist hair strands were then placed directly after the coloring under a UV analysis light with two radiation sources.

The first radiation source is a low-pressure mercury vapor discharge lamp. The lamp emits UV-C radiation with a peak value of 253.7 nm (UV-C). At least 85% of the energy of this lamp is converted into UV light having a wavelength of 253.7 nm.

The second radiation source is a fluorescent tube which emits UV radiation in the range of 350-400 nm with a maximum at 368 nm.

Both radiation sources were switched on, and the colored hair strands were irradiated with the light of both radiation sources for 20 minutes. The hair strands were then measured again colorimetrically.

The colored strands used for the comparison were colorimetrically measured without irradiation and used in the tests for wash-fastness.

### 3. Wash-Fastness

For the measurement of the wash-fastness, the strands were then washed 3 times or 10 times.

For each hair wash, a commercially available shampoo (0.25 g shampoo (Schauma 7 Herbal) per 1 g of hair) was applied to the strand, and the shampoo was massaged in with the fingers for 30 seconds. The shampoo was then rinsed out for 1 minute under running lukewarm water, and the hair strand was dried. The process described above corresponds to hair washing. For each additional hair wash, the process was repeated. The hairs washed in this way were measured colorimetrically.

The dE value used for the assessment of the color retention is derived from the L*a*b* colorimetric values measured at the respective strand as follows:

$$dE = {[ {( {L}_{i} - {L}_{0} )}^{2} + {( {a}_{i} - {a}_{0} )}^{2} + ( {b}_{i} - {b}_{0} ) ]}^{1 / 2}$$

L₀, a₀, and b₀=measured values of the uncolored strand
Lᵢ, aᵢ, and bᵢ=measured values of the colored or colored and washed strand

The smaller the dE value, the lower the color distance between the uncolored and the colored (or colored and washed hair). The greater the dE value, the higher the color difference compared to the uncolored strand.

The dL value used for the assessment of brightness is derived from the L value measured at the respective strand as follows:

$$dL = {[ {( {L}_{i} - {L}_{0} )}^{2} ]}^{1 / 2}$$

L₀=measured value of the uncolored strand
Lᵢ=measured value of the colored or colored and washed strand

The greater the dL value, the greater the brightness difference compared to the uncolored strand.

The following color results were obtained

| Color distance dE between | dE value | dE value | dE value |
|---|---|---|---|
| uncolored and colored/ | 0 hair | 3 hair | 10 hair |
| washed strand strands | washes | washes | washes |
| Coloring with (F) without | 63.16 | 53.85 | 46.01 |
| irradiation | | | |
| Coloring with (F) and 20 | 62.97 | 60.25 | 53.24 |
| min irradiation | | | |

Directly after coloring (0 hair washes), the comparative strand and the irradiated strand had in principle the same color distance from the uncolored hair, i.e., both strands were colored with the same color intensity.

After 3 hair washes and after 10 hair washes, however, the irradiated hair strand had a significantly greater color distance from the uncolored hair, which means that the wash-fastness of the colored strand could be massively improved by the irradiation.

| Brightness difference dL | dL value | dL value | dL value |
|---|---|---|---|
| between uncolored and | 0 hair | 3 hair | 10 hair |
| colored/washed strand | washes | washes | washes |
| Coloring with (F) without | 41.66 | 34.70 | 28.01 |
| irradiation | | | |
| Coloring with (F) and 20 | 40.63 | 37.67 | 32.56 |
| min irradiation | | | |

Directly after coloring (0 hair washes), the comparative strand and the irradiated strand had in principle the same brightness distance from the uncolored hair.

After 3 hair washes and after 10 hair washes, however, the irradiated hair strand had a significantly greater brightness distance from the uncolored hair. The improvement in the wash-fastness of the colored strand by irradiation was therefore also found in the dL values.

What is claimed is:

1. A method for coloring human hair, comprising
(1) applying a coloring agent (F) to hair which comprises
(f1) at least one amino-functionalized silicone polymer, and
(f2) at least one pigment, and
(2) irradiating the hair in step (1) for a period of 1 to 60 minutes with UV/VIS radiation in the wavelength range of 200 to 800 nm.

2. The method according to claim 1, characterized in that the coloring agent (F) comprises at least one amino-functionalized silicone polymer (f1) which comprises at least one structural unit of the formula (Si-amino), 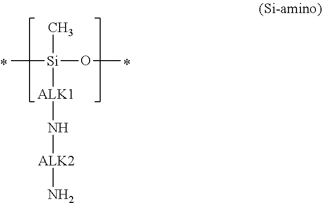
where ALK1 and ALK2 represent, independently of one another, a linear or branched, divalent C₁-C₂₀ alkylene group.

3. The method according to claim 1, wherein the coloring agent (F) comprises at least one amino-functionalized silicone polymer (f1) comprising structural units of the formula (Si-I) and of the formula (Si-II)

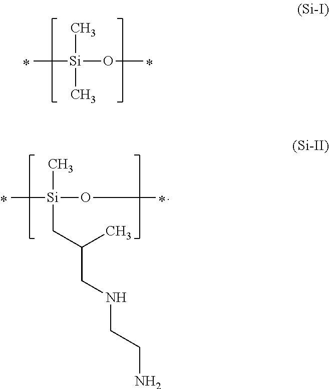

4. The method according to claim 1, wherein the coloring agent (F) comprises—relative to the total weight of the coloring agent (F)—one or more amino-functionalized silicone polymers (f1) in a total amount of 0.1 to 20 wt %.

5. The method according to claim 1, wherein the coloring agent (F) comprises—relative to the total weight of the coloring agent (F)—one or more amino-functionalized silicone polymers (f1) in a total amount of 0.3 to 5 wt %.

6. The method according to claim 1, wherein the coloring agent (F) comprises—relative to the total weight of the coloring agent (F)—one or more amino-functionalized silicone polymers (f1) in a total amount of 0.5 to 2.0 wt %.

7. The method according to claim 1, wherein the coloring agent (F) comprises at least one inorganic pigment (f2), wherein the inorganic pigment is selected from the group consisting of colored metal oxides, metal hydroxides, metal oxide hydrates, silicates, metal sulfides, complex metal cyanides, metal sulphates, bronze pigments, and mica-based colored pigments which are coated with at least one metal oxide and/or metal oxychloride.

8. The method according to claim 1, wherein the coloring agent (F) comprises at least one organic pigment (f2), wherein the organic pigment is selected from the group consisting of carmine, quinacridone, phthalocyanine, sorghum, blue pigments having the Color Index Numbers, Cl 42090, CI 69800, CI 69825, CI 73000, CI 74100, CI 74160, yellow pigments having the Color Index Numbers, CI 11680, CI 11710, CI 15985, CI 19140, CI 20040, CI 21100, CI 21108, CI 47000, CI 47005, green pigments having the Color Index Numbers, CI 61565, CI 61570, CI 74260, orange pigments having the Color Index Numbers, CI 11725, CI 15510, CI 45370, CI 71105, red pigments having the Color Index Numbers, CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, and CI 75470.

9. The method according to claim 1, wherein the coloring agent (F) contains at least one pigment (f2) selected from the group consisting of pigments based upon a lamellar substrate platelet, pigments based upon a lenticular substrate platelet, and vacuum-metallized pigments.

10. The method according to claim 1, wherein the coloring agent (F) comprises, relative to the total weight of the coloring agent (F), one or more pigments (f2) in a total amount of 0.01 to 10.0 wt %.

11. The method according to claim 1, wherein the coloring agent (F) comprises, relative to the total weight of the coloring agent (F), one or more pigments (f2) in a total amount of 0.2 to 2.5 wt %.

12. The method according to claim 1, wherein the coloring agent (F) comprises, relative to the total weight of the coloring agent (F), one or more pigments (f2) in a total amount of 0.25 to 1.5 wt %.

13. The method according to claim 1, wherein the, following applying the coloring agent (F) to the hair, the coloring agent is left on the hair for a period of 1 to 30 minutes after which the coloring agent is rinsed out with water and the irradiating of the hair occurs for a period of 1 to 60 minutes with an artificial light source which emits UV/VIS radiation in the wavelength range of 200 to 800 nm.

14. The method according to claim 13, wherein a period of a maximum of 72 hours passes between rinsing the coloring agent from the hair and irradiating the hair.

15. The method according to claim 1, wherein the irradiation (2) of the hair is for a period of 2 to 50 minutes with an artificial light source which emits electromagnetic radiation having wavelengths in the range of 200 to 800 nm.

16. The method according to claim 1, wherein the irradiation (2) of the hair is for a period of 10 to 30 minutes with an artificial light source which emits electromagnetic radiation having wavelengths in the range of 200 to 800 nm.

17. The method according to claim 1, wherein the (2) irradiation of the hair is with UV-A radiation having a wavelength of 400 nm-320 nm, UV-B radiation having a wavelength of 320 nm-280 nm, and/or UV-C radiation having a wavelength of 280 nm-200 nm.

18. The method according to claim 1, wherein the irradiation (2) is carried out using a gas discharge tube, a gas discharge lamp, a light-emitting diode, a pulsed xenon lamp, and/or an excimer lamp.

19. The method according to claim 1, wherein the irradiation (2) is carried out using an artificial radiation source which converts at least 60% of its energy into UV-C radiation having a wavelength of 250 nm to 260 nm.

20. The method according to claim 1, wherein the irradiation (2) is carried out using an artificial radiation source which converts at least 60% of its energy into UV-A radiation having wavelength of 350 nm to 380 nm.
